(19) United States
(12) Patent Application Publication
(10) Pub. No.: US 2018/0285153 A1
(43) Pub. Date: Oct. 4, 2018

(54) MANAGING A MICROFLUIDIC DEVICE

(71) Applicant: HEWLETT-PACKARD DEVELOPMENT COMPANY, L.P., Houston, TX (US)

(72) Inventors: Joshua M. Yu, Corvallis, OR (US); Matthew David Smith, Corvallis, OR (US); Manish Giri, Corvallis, OR (US); Shameed Salt M A, Bangalore (IN)

(73) Assignee: HEWLETT-PACKARD DEVELOPMENT COMPANY, L.P., Houston, TX (US)

(21) Appl. No.: 15/772,463

(22) PCT Filed: Feb. 4, 2016
(86) PCT No.: PCT/US2016/016582

§ 371 Date: Apr. 30, 2018

(51) Int. Cl. G06F 9/48; G01N 35/00; G06F 9/54; G16H 50/20; B01L 3/00

(52) U.S. Cl. CPC G06F 9/4881; G01N 35/00584; G06F 9/542; G16H 50/20; B01L 2300/02; G01N 2035/00158; G01N 2035/00881; G01N 2035/00891; B01L 3/502715

(57) ABSTRACT

A system and method for managing a microfluidics device. The system includes a microfluidics device; a controller with a first processor which receives outputs from the microfluidics device and provides inputs to the microfluidics device; and a computing device with a second processor and an application program interface (API). The computing device provides instructions to the controller using the API. The instructions are executed by the first processor to produce real-time outputs to the microfluidics device.

### BACKGROUND

Point of Care (POC) refers to the emerging field of providing diagnostic or other testing where the patient is located, generally with the understanding that test results will be available while the medical professional and the patient are still together. This contrasts with the more traditional model of using a central lab and sending biological or other samples to be tested and then receiving the information back at some later period in time.

### BRIEF DESCRIPTION OF THE DRAWINGS

The accompanying drawings illustrate various examples of the principles described herein and are a part of the specification. The illustrated examples are merely illustrative and do not limit the scope of the claims. Like numerals denote like but not necessarily identical elements.

FIG. 1 shows a system for performing microfluidics measurements consistent with one example described in this specification.

FIG. 2 shows a system for performing microfluidics measurements consistent with one example described in this specification.

FIG. 3 shows a method for performing microfluidics measurements consistent one example described in with this specification.

FIG. 4 shows a computer-readable storage medium consistent with one example described in this specification.

### DETAILED DESCRIPTION

Challenges to developing POC diagnostic tests have fallen in two major categories: technical and economic. Technical challenges have included developing and refining tests, limiting the number of reagents used, minimizing the size of the equipment to run the tests to render them portable, creating versions of the test that are shelf stable, etc. Economic challenges tend to relate to the relatively expensive costs of equipment to run the tests, the sunk cost advantages of the existing lab format, etc. Nevertheless, the advantages to a medical professional to obtain information to make a diagnosis on the spot and begin treatment without requiring a second appointment make the POC approach attractive. There have accordingly been ongoing efforts to convert traditional lab services to P00 accessible tests.

One model used in POC devices involves a cartridge, chip, or similar component that is attached to a second device. In this specification and the attendant claims, the term cartridge is used broadly to include chips and similar components that are removably associated with a device in order to conduct a microfluidics test. The cartridge may be disposable. Alternately, the cartridge may be reusable. The cartridge may be preloaded with the reagents to perform tests. The cartridge, thus, contains the materials to run the test but may be changed out between testing, while the second device is used with multiple cartridges to perform multiple tests. If the cartridge is disposable, the cost of producing the cartridge may ultimately drive the cost of the test. If the cartridge is reusable, there may be more flexibility in terms of costly components in the cartridge. But the cost of the cartridge, including the number of times it can be recycled, may impact the cost of performing the test.

One area that has contributed to reducing the cost of the cartridge is the emergence of microfluidics testing. Microfluidics refers to processes designed to operate on small fluid volumes, often nanoliter (n1) or picoliter (p1) volumes. Such small fluid volumes do not behave the same as larger scale fluid volumes. This is driven, in part, by the large surface area to volume ratios involved in microfluidics. Gravity and inertial forces generally play a much smaller role, while surface energy and capillary effects are more pronounced, compared with larger sized (e.g. ml or microliter) volumes. The size of materials suspended in the fluid becomes large relative to the channels, enabling other technologies, for example light based detection or shape analysis of individual cells. Heat and mass transfer phenomenon are similarly changed relative to macro scale events. Microfluidics also enables measurements that would be difficult or expensive to obtain using other methods.

Microfluidics offers a number of advantages over larger scale test methods. Microfluidics may use small samples, which makes obtaining a sample less painful and troublesome for the patient. It also may allow multiple independent measurements to be made from a small volume of material. This can increase the reliability of measurements using averaging of replicate tests performed using a single cartridge. This may have the corresponding challenge of being more vulnerable to variations in the sample. Microfluidics methods may also use correspondingly smaller quantities of test reagents. Microfluidics methods also may tend to be much less dependent on and vulnerable to gravity. This is both an advantage and a disadvantage as gravity is often used for separations in chemical and biological testing. However, gravity independence can be helpful in a handheld or portable device that may be used in a variety of orientations. In contrast, a large piece of lab equipment will probably maintain only a fixed orientation.

Microfluidic based testing has benefited from the development of precision manufacturing techniques for Micro Electrical Mechanical Systems (MEMS). MEMS combine electronics with mechanical and/or other components to create small scale devices. While operating on this small scale has made making devices a challenge, it may have also reduced the material and manufacturing costs. Further, MEMS technologies have been able to adapt many technologies, techniques, and materials from the electronics industry. Because features on MEMS are often much larger than the minimum line width in circuits, MEMS can often be manufactured economically with established technologies.

As noted above, fluid dynamics associated with lab level sized equipment does not always scale down to microfluidics. New models have been and continue to be developed to understand, measure, and predict fluid behavior in small channels where the surface effects have a much larger contribution compared with the bulk properties. In some cases, this has produced new measurement techniques or different ways to characterize samples. For example, at a macro scale, to separate a solution by size has involved pumping the solution through a prepared column under controlled conditions and using included controls. In some microfluidics applications, the routing channel between the sample and the test location can be used to produce this effect and remove contaminants or other continuants that may increase the noise in the measurement.

The specification describes a system and method for performing measurements and/or tests using a microfluidics device. The method has the specific advantage over previous methods in that it reduces the processing power and therefore the cost of the firmware/hardware associated with the testing device by using a second processor to provide much of the processing power for calculations and user interaction. Thus, a processor in the POC device provides basic control functions and data gathering from the fluidic device in real time while using the second processor to perform the processor intensive activities in an asynchronous manner. This reduces the costs associated with the microfluidics device and its controller. The described approach also facilitates modularity in the fluidics device and the user interface which allows faster iteration of the technology while allowing backwards compatibility and limiting of the need to replace hardware, thereby reducing costs.

Accordingly, the present specification describes, among other examples, a system for managing a microfluidics device. The system comprises: a microfluidics device: a controller comprising a first processor, the first processor to receive outputs from the microfluidics device and the first processor to provide inputs to the microfluidics device; and a computing device comprising a second processor and an application program interface (API), wherein the controller is to receive instructions from the computing device and the instructions are executed by the first processor to produce real-time outputs to the microfluidics device.

The present specification also describes a method for off-loading tasks from a first processor managing a microfluidics device. A method for off-loading tasks from a first processor managing a microfluidics device, the method comprising: in response to receiving an operation at the first processor, obtaining a measurement from the microfluidics device; and providing the measurement to a second processor for processing to decrease an impact on performance of the first processor.

The present specification also describes a non-transitory, computer-readable storage medium comprising computer readable instructions. When the instructions are executed, the instructions cause a group of processors to: A non-transitory, computer-readable storage medium comprising computer readable instructions which when executed cause a group of processors to: receive a request at a second processor; provide an instruction from the second processor to a first processor; and receive an output from the first processor to the second processor, wherein the output from the first processor to the second processor comprises information from an output of a microfluidics device measured by the first processor.

Turning now to the figures:

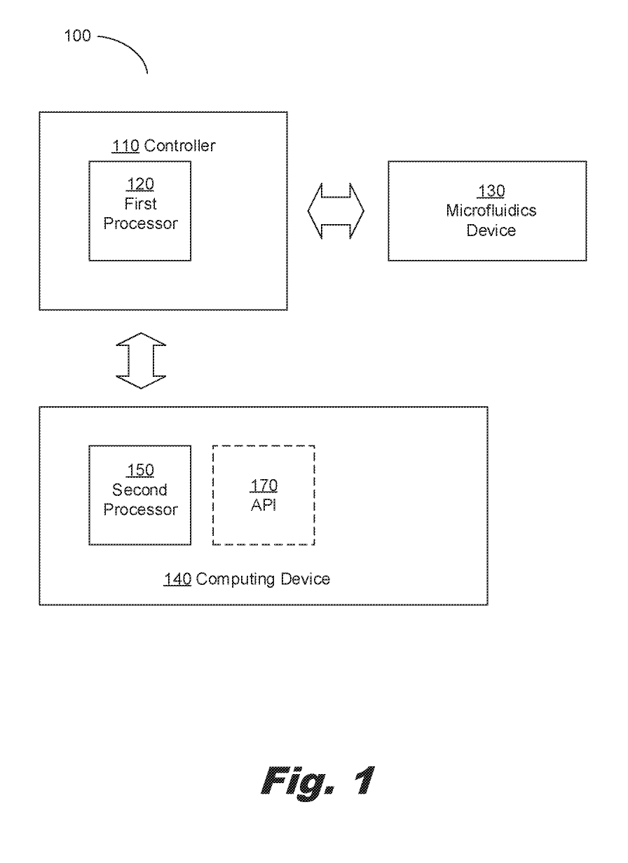

FIG. 1 shows a system (100) for performing microfluidics measurements consistent with this specification. The system (100) comprises a controller (110) with a first processor (120), a cartridge (130) containing systems and materials to perform a microfluidic test, and a computing device (140) comprising a second processor (150). The first processor (120) is functionally in communication with the second processor (150). The first processor (120) is functionally connected with the system of the cartridge (130) for performing the microfluidics test. The first and second processors (120, 150) may both have locally stored instructions. Alternately, the second processor (150) may have locally stored instructions and provide instructions to the first processor (120). Finally, the first and second processors (120, 150) may both rely on provided instructions, for instance downloaded from a server, in order to conduct operations.

The system (100) is for performing a microfluidic measurement. In some examples, the system (100) performs multiple measurements, either simultaneously and/or serially. The system includes a controller (110) which may be thought of as the device controlling the measurements. The system also includes a cartridge (130) which may or may not be disposable. The use of cartridges (130) facilitates rapid testing as well as providing a variety of functionality for the system. For example, one type of cartridge (130) allows a first type of microfluidics test, while a second type of cartridge (130) allows a second type of microfluidics test. The system also makes use of a computing device (140). The computing device (140) allows the system (100) to move computation. translation, and other functions from the controller (110) to a second processor (150) in the computing device (140). This reduces the costs associated with the controller (110), which has a significant impact on the ability to apply the test method as a point of care test.

The controller (110) is a piece of hardware that controls the test performed on the cartridge (130). In some examples, the controller (110) is a disposable. In other examples, the controller (110) is retained and used for multiple tests. The controller (110) may be a durable medical device that is used for multiple sets of tests.

The first processor (120) is located in the controller (110). In an effort to reduce the cost of the cartridge (130), some designs have increased the size and complexity of the first processor (120) controlling the operations on the cartridge (130). While this approach has the advantage of reducing the cost of the cartridge (130) and thereby reducing the per test cost, it has also increased the cost of the controller (110). In contrast, the approach described herein also minimizes the processing power and the firmware/hardware in the first processor (120). This reduces the cost of the controller (110), making it cheaper to provide to users and limiting the cost in the event of loss/damage. Because the controller (110) is expected to be used at the point of care, the controller (110) is also more likely to be lost or damaged compared to a convention and non-portable piece of testing equipment, such as a Mass-Spectrometer (MS) or an autotitrator in a lab environment. Further, the move to the POC approach also implies the use of a larger number of controllers (110) to perform the tests that would previously have been performed on a single, centralized piece of lab equipment. A POC approach is unlikely to be as cheap as a lab based approach since a POC approach uses multiple controllers (110), while a lab based approach can share a single controller (110). However, reducing the cost of the controller (110) helps enable POC. In considering POC, the tradeoff becomes the value of obtaining the testing result while with the patient vs. the incremental costs of the additional controllers (110). Thus, reducing the controller (110) cost facilitates adoption of POC.

The cartridge (130) contains the actual components for performing a microfluidics based test. In some examples, the cartridge (130) is capable of performing a plurality of instances or runs of a given test sequentially and/or serially on a single and/or multiple samples. The cartridge (130) may be capable of performing multiple kinds of microfluidics tests. The specific methodologies and techniques to perform the tests are not the focus of this specification. However, examples of such tests include: glucose testing, coagulation, cytology, cardiac markers and detection of infectious diseases.

The cartridge (130) communicates with the first processor (120) on the controller (110). In one example, this is accomplished by electrical conductors. The conductors may pass through a port that facilitates connection between the cartridge (130) and the controller (110). The port may stabilize the cartridge relative to the controller. The port may use an existing standard, for example, the various Universal Serial Bus (USB) standards. The port may also be a custom configuration. The port may include conductors and/or contacts that are used with a single type of cartridge (130) and are not used with a second type of cartridge (130). In one example, the configuration of contacts on the cartridge (130) facilitates identification of the cartridge (130) type by the controller (110). The port may transfer signals between the controller (110) and cartridge (130). The port may transfer power between the controller (110) and the cartridge (130). In one example, the controller (110) includes a battery, which powers the operation on the cartridge (130). In a second example, the cartridge (130) includes a battery to power the controller (110) and/or the cartridge (130). The communication between the controller (110) and the cartridge (130) may be conducted by alternate methodologies including commonly available standards such as Bluetooth and Wi-Fi. Further, any suitable method of short range communication including light, induction, electromagnetic, radio, and/or directed electrical contact may be used.

The computing device (140) comprises the second processor (150). The computing device (140) can be any suitable computing device, for example, a phone, a tablet, a pad, a laptop, a desktop, a server, a dedicated device, or a general purpose computer. The rapid and continuing increase in available processor power in portable devices that are being carried by large numbers of individuals provides the opportunity to 1) reduce the capabilities of the first processor (120) and 2) take advantage of the continued growth in available processor power without having to engage in frequent hardware updates to the controller (110). These two factors in turn, reduce the cost of the system (100) generally and specifically reduce the cost of the controller (110) and cartridge (130), allowing high quality data acquisition and processing with low unit cost. This helps the system (100) achieve a manufacturing cost that will encourage adoption and use as a point of care diagnostic.

The computing device (140) can include a display. The computing device (140) can include a device for a user to provide commands, for example, a mouse, touch screen, touch pad, keyboard, stylus, and/or similar devices. By moving display and interface operations from the controller (110) to the computing device (140), the cost of the controller (110) can be reduced. Similarly, in some examples, the computing device (140) provides power to the controller (110). This can be accomplished using a wired connection, for example, a USB port and/or other standardized power connection available on general purpose computing devices (140). The computing device (140) may have an internal battery and/or receive power from an external power source.

The computing device (140) may include a memory. Alternately, the memory may be associated with the controller (110) and/or a remote system, for example, on a server.

The second processor (150) can be a conventional processor. The second processor (150) may be a dedicated system with specifically designed firmware. More likely, the second processor (150) is part of a general purpose computing device that can be programmed to support the first processor (120) by performing activities that are either outside the capabilities of the first processor (120) or would impact the performance or cost of the first processor (120). In one example, the second processor (150) handles interrupts and/or multiple threads and thus does not have the ability to guarantee the timing of output pulses and control signals to make the cartridge perform the desired functions at a desired timing. In contrast, the first processor (120) may have real time output control so as to be able to precisely regulate the timing of provided and received signals to the cartridge (130). In one example, the first processor is to provide timed firing sequences to the microfluidics device. Accordingly, even though the second processor (150) may be more powerful than the first processor (120), as used, the first processor (120) has at least one feature that is unavailable in the second processor (150). In order to use the second processor (150) to provide the desired support for the first processor (120), it is converted from a general purpose machine to specific machine with coded instructions to manage the exchange and processing of information.

In some examples, the second processor (150) also facilitates display and control of the system (100) operations. The second processor (150) may just serve as support for the first processor (120). Alternately, the second processor (150) may provide other functions, for example, making calls to an API (170) available to the second processor (150). The second processor (150) may operate other software that controls the interface and other functions.

The use of an application programming interface or API (170) provides numerous advantages. The API (170) provides an interface between external systems and the controller (110). This allows the API (170) to deal with changes to the controller (110), first processor (120), and/or cartridge (130) rather than having to make changes in the external software for any system improvements. It also facilitates independence of the first processor (120) configuration on data provided by the system (100). For example, the first processor (120) may make measurements in terms of clock cycles, however, the API (170) can identify the clock speed of the first processor (120) and convert the output data into Hz or a duration. Thus, an external system interacting with the system (100) does not need to be concerned with the particular hardware being used but rather can make requests using a set of preprogrammed operations that return information in a controlled format independent of the generation of the first processor (120) and/or microfluidics device (130). This facilitates independence of the external software development from the hardware allowing improvement in both areas to operate independently by using the API (170) to provide the interface.

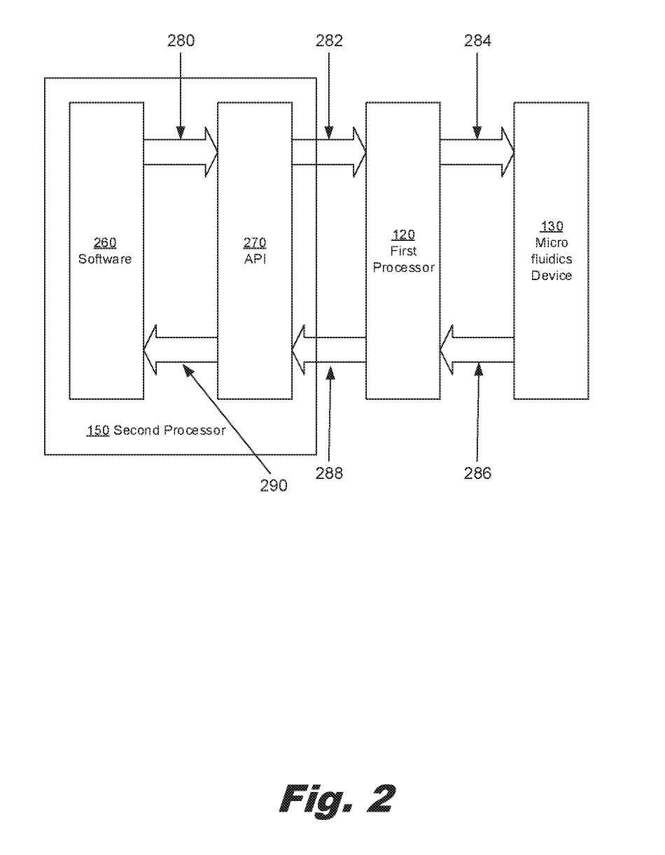

FIG. 2 shows a system for performing microfluidics measurements consistent with this specification. The system includes the first processor (120) and the cartridge (130) as well as the second processor (150) which is part of a computing device (140). Inside the second processor (150) there is both a software (260) and an application programming interface (API) (270). FIG. 2 also shows information moving between these portions of the system. The software (260) provides instructions (280) to the API (270) which converts the instructions (280) into operations (282) provided to the first processor (120). The first processor (120) executes the operations (282) and provides inputs (284) to the cartridge (130). The first processor (120) obtains outputs (286) from the cartridge (130). The first processor (120) then provides these outputs (286) with or without modification to the API (270) as measurements (288). The API (270) processes the measurements (288) and provides them to the software (260) as return variables (290). As used in this specification and the attendant claims, return variables (290) includes passing information by value as well as by location (e.g., pointer, register, or memory).

The software (260) is shown as operating on the second processor (150) associated with the computing device (140). However, other arrangements are possible. The software (260) may be operated on a third processor and make calls to the API (270). The API (270) may, instead of providing the instructions directly to the first processor (120), relay the instructions through the third processor. For example, the second processor (150) may be a server or similar device accessible through a network, such as the internet. This allows calls to ensure that the API (270) is up to date and avoids the potential use of a non-updated version of the API (270). Separating the instructions (280) provided by the software (260) from the operations (282) provided to the first processor (120) allows the software (260) to be written independent of the specification of the first processor (120). This allows the software (260) and the hardware including the controller (110), first processor (120), and the cartridge (130). to be managed using the API (270).

The use of an API (270) provides numerous advantages. The API allows the instructions of the software (280) to be organized as calls to the API (270). This allows the software (280) and the rest of the system (100) to be decoupled such that different parts of the system (100) can be improved individually. The API (270) translates the instructions (280) from the software (260) into operations (282) that are provided to the first processor (120). Accordingly, if the first processor (120) is upgraded and/or modified, the API (270) can be adjusted without impacting the software (260). The API (270) can also be designed to provide different instructions based on the model of controller (110), first processor (120), and/or the cartridge (130). This makes it easier to maintain backwards compatibility.

Similarly, the API (270) receives the measurements (288) from the first processor (120). The API (270) can then convert the measurements into other formats prior to providing them to the software (260). In one example, the API (270) removes the first processor (120) specific elements from the measurements (288) and converts them to standard formats. For example, the measurements (288) may be provided in terms of clock counts which depend on a clock used by the first processor (120). The API (270) converts the measurements (288) from clock counts into time or frequencies. The information from the measurements (288) can then be provided to the software (260) in a form that is independent of the first processor (120) and/or cartridge (130).

The software (260) provides instructions (280) to the API (270). The instructions (280) may include testing operations on the microfluidic device (130) that are made accessible to the software (260). While, in theory, the list of instructions could include every possible operation that may be performed by the controller (110) and the cartridge (120), in practice, the available instructions represent a subset of possible operations. The instructions (280) may include routines, including complex routines up to and including conducting a single or multiple tests using the cartridge (130). For example, a sample instruction of GlucoseTest( )may cause the API to instruct the first processor to perform a glucose test on a loaded and prepared cartridge (130). Instructions (280) may be formatted in any suitable language or coding schema including, but not limited to: text, high level language, machine code, memory calls, etc. Other example instructions (280) may include obtaining serial or configuration data for the cartridge (130), first processor (120), controller (110), and/or the API (270).

Instructions (280) do not need to produce activity in the cartridge (130). For example, the instructions (280) may request the API (270) to perform processing on previously obtained measurements. Similarly, an instruction (280) to obtain a version number of the API (270) does not involve communication with the first processor (120).

The operations (282) are provided by the API (270) to the first processor (120) on the controller (110). The operations (282) are machine level instructions processed by the first processor in order perform the tasks outlined in the instructions (280). The API (270) converts the instructions (280) into the operations (282) such that the operations are compatible with the first processor (120), controller (110), and the cartridge (130).

The inputs (284) are provided by the first processor (120) to the cartridge (130). These include, for example, control signals, firing signals, routing signals, and similar communication. Some of the inputs (284) may be provided as binary signals while others may be provided as analog signals. The inputs allow the cartridge to perform the operations to perform a microfluidics test or measurement.

Similarly the first processor (120) receives outputs (286) from the cartridge (130). The outputs (286) may be time sensitive or time invariant signals from the cartridge (130). The outputs (286) may be digital and/or analog.

The measurements provided by the first processor (120) may include processing from the outputs (286) received by the first processor (120). However, it is preferable to minimize the operations performed by the first processor (120) that can be performed by the second processor (150). This minimizes the capacity for the first processor (120), which reduces the cost of the controller (110). This, in turn, impacts the ability to provide the testing as point of care rather than by a more traditional, lab based approach.

However, nothing in this disclosure prevents operations by the first processor in reformatting, calculating, correcting, and/or otherwise modifying the outputs (286) prior to providing the information in them as measurements (288) to the second processor (150). In some instances, it may be possible to perform some operations on the first processor (120) without impacting other operations by the first processor (120). Further, the price floor and/or assurance of supply for low end processors capable of providing the control capabilities for conducting may result in the first processor (120) providing some operations.

The return variables (290) output by the API (270) to the software (260) will generally be subjected to processing by the API (270). As discussed above, while it is possible to arrange for the software (260) to have access to all the outputs (286), such an approach prevents the software from being written in a manner that is independent of the particular controller (110), first processor (120), and/or cartridge (130) used. Accordingly, it gives up some of the benefits of the API (270) in translating between the software (260) and the hardware. Such an approach still retains the real-time advantages of the first processor (120).

In contrast, the use of the API (270) to process the measurements provides significant advantages in offloading operations from the first processor (120) to the second processor (150). Because the second processor is part of a computing device (140) that can be used for a variety of purposes, the processing power of the computing device (140) is in some senses “free” as it likely represents a very low incremental cost to perform the described processing. The processing performed in the API (270) can be performed as resources become available and can be operated as a low priority thread or application on the computing device (140). By using the underutilized resources of the computing device (140), the system can drastically improve the performance of the system (100) as a whole, reduce the costs of the controller (110) due to the first processor (120), and enable cost effective provision of point of care testing. Point of care testing, in turn, may provide better outcomes for patients by reducing the time to begin treatment or by avoiding incorrect treatment based off of non-data approaches.

Further, the continued increase in processing power available from computing devices (140), especially, phones, tablets, laptops, and similar highly portable devices implies that the system (100) can be expected to improve performance without having to change the controller (110) or cartridge (130) designs. This can be expected to increase the useful lifetime of the controller (110). Longer controller (110) lifetimes can further reduces the cost per test.

In addition to performing data processing on the second processor (150), the API (270) may also convert the data into forms that are independent of the characteristics of the specific first processor (120) and/or cartridge (130) used. Accordingly, the API (270) functions to translate between the hardware and the software (260) allowing the software (260) to be independent of the specific hardware used to perform the testing. This facilitates the independent development and improvement of the software (260) from the controller (110) and/or cartridge (130). Translation by the API of the output variables to standardized units, such as Hz, volts, seconds, etc. facilitates presentation and/or analysis in the software (260). Thus, the software (260) can include features specific to the computing device, for example by focusing on input/output or test automation. The software (260) may also provide for display or output of the test results.

While this specification has used different terms for the elements 280, 282, 284, 286, 288, and 290, it will be apparent to one of ordinary skill in the art that these categories of information and instruction transfer are not exclusive but rather can overlap to a considerable degree. Accordingly, because something is described as possible with respect to one more of these types of information transfer it is not intended to exclude that same element from the other types of information transfer discussed.

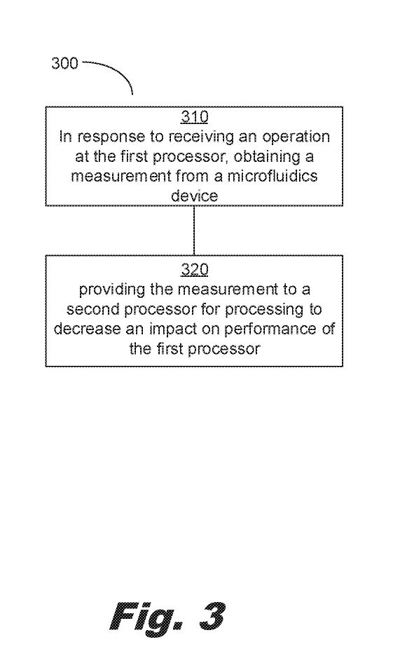

FIG. 3 shows a method (300) for off-loading tasks from a first processor managing a microfluidics device consistent with this specification. This off-loading facilitates the use of a processor with reduced complexity. The method (300) comprises, in response to receiving an operation at the first processor (120), obtaining a measurement from a microfluidics device (130) (310) and providing the measurement to a second processor (150) for processing to decrease an impact on performance of the first processor (120) (320).

Operation (310) includes receiving an operation at a first processor (120) and in response obtaining a measurement from a microfluidics device (130). The first processor (120) capabilities and cost are minimized by having the operation provided. Reducing the cost of the first processor (120) facilitates lower testing cost and enables point of care testing. The first processor (120) obtains the measurement from the microfluidics device (130). In some examples, the first processor converts the measurement from analog to digital. In some examples, the first processor (120) reports the measurement in terms of first processor (120) specific units, for example, clock ticks.

Operation (320) includes providing the measurement to a second processor (150) for processing to decrease an impact on performance of the first processor (120). The use of a second processor (150) to perform the processing operations on the measurement reduces the cost of the first processor (120). The second processor (150) can be used on a temporary basis when testing is being performed but may be used for other purposes simultaneously with testing or when testing is not being performed. This allows a general purpose computer such as one from a smartphone, tablet, desktop, laptop, or similar device to provide the processing power to enable testing. Using other devices to provide the data processing allows the capabilities and cost of the first processor (120) to be kept low. This facilitates a low cost controller (110) which supports adoption of point of care testing.

In one example, the data is processed by the second processor (150) and the output of the processed data is used to provide an instruction to perform a second test on the microfluidics device (130). The system may conduct a series of tests, analyzing the results after each test, until the accumulated results reach a certain confidence interval with respect to one or more testing levels. For example, replicates of a total protein test could be repeated until a 95% confidence interval was obtained with respect to a high protein action level. In one example, this activity is conducted automatically using replicate test setups on the microfluidics system that use a same sample provided to the microfluidics system. In another example, the system prompts a user for an additional sample. The system may conduct a second test based on the outcome of the first test. For example, if a calculated first test result obtained by the second processor (150) is outside of a predetermined range, the system may automatically conduct a second microfluidics test on the same sample or an additional sample. The second microfluidics test may be a different microfluidics test from the first microfluidics test. The second microfluidics test may be a replicate of the first microfluidics test.

The measurement may be provided to the second processor (150) as measured by the first processor (120). The measurement may be provided to the second processor (150) after some initial processing by the first processor (120). The measurement may be provided in terms of first processor specific units, for example, clock ticks. The first processor (120) may lack the capability to perform subsequent data processing of the measurement that is performed by the second processor (150).

While the claimed method (300) comprises these operations, additional operations may also be included. For example, the first processor (120) may obtain a plurality of measurements from the microfluidics device (130) and provides the plurality of measurements to the second processor (150) for processing. The second processor (150) may output the measurement to a display. The second processor (150) may store the processed measurement to a computer readable format. The measurement of the microfluidics device (130) may be selected from a resistance, an impedance, a conductivity, a concentration, a temperature, a voltage, an absorption, a transmission, a fluorescence, and/or an amount of light scattering.

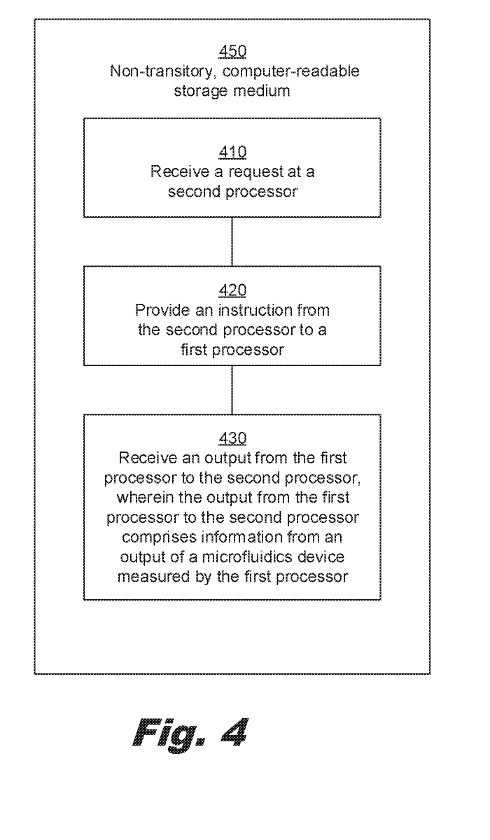

FIG. 4 shows a non-transitory, computer-readable storage medium (450) comprising computer readable instructions which, when executed, cause a group of processors to perform the following operations: receiving a request at a second processor (150) (410); providing an instruction from the second processor (150) to a first processor (120) (420); receive an output from the first processor (120) to the second processor (150), wherein the output from the first processor (120) to the second processor (150) comprises information from an output of a microfluidics device (130) measured by the first processor (430).

Within the principles described by this specification, a vast number of variations exist. The examples described are just examples, and are not intended to limit the scope, applicability, or construction of the claims in any way.

What is claimed is:

1. A system for managing a microfluidics device, the system comprising:
a microfluidics device;
a controller comprising a first processor, the first processor to receive outputs from the microfluidics device and the first processor to provide inputs to the microfluidics device; and
a computing device comprising a second processor and an application program interface (API), wherein the controller is to receive instructions from the computing device and the instructions are executed by the first processor to produce real-time outputs to the microfluidics device.

2. The system of claim 1, wherein the first processor is to task the second processor with data processing tasks.

3. The system of claim 1, wherein the first processor is to provide timed firing sequences to the microfluidics device.

4. The system of claim 1, wherein the first processor is to receive a series of commands from the second processor that results in a plurality of outputs from the first processor to the microfluidics device.

5. The system of claim 1, wherein the first processor is to convert an instruction received from the second processor to plurality of signals provided to the microfluidics device.

6. The system of claim 1, wherein the first processor is to provide a plurality of data measurements from the microfluidics device to the second processor in response to the first processor receiving an instruction from the second processor.

7. The system of claim 1, wherein the second processor is to convert information received into a user presentable format.

8. A method for off-loading tasks from a first processor managing a microfluidics device, the method comprising:
in response to receiving an operation at the first processor, obtaining a measurement from the microfluidics device; and
providing the measurement to a second processor for processing to decrease an impact on performance of the first processor.

9. The method of claim 8, wherein, in response to receiving the instruction, the first processor obtains a plurality of measurements from the microfluidics device and provides the plurality of measurements to the second processor for processing.

10. The method of claim 8, wherein the first processor provides the measurement to the second processor without modification.

11. The method of claim 8, wherein the first processor provides the measurement to the second processor in terms of clock counts of the first processor.

12. The method of claim 8, wherein the measurement of the microfluidics device is selected from: a resistance, an impedance, a conductivity, a concentration, an absorption, a voltage, a temperature, a transmission, fluorescence, and an amount of light scattering.

13. A non-transitory, computer-readable storage medium comprising computer readable instructions which when executed cause a group of processors to:
receive a request at a second processor;
provide an instruction from the second processor to a first processor; and
receive an output from the first processor to the second processor, wherein the output from the first processor to the second processor comprises information from an output of a microfluidics device measured by the first processor.

14. The computer-readable storage medium of claim 13, wherein the request at the second processor results in output from the first processor to the second processor of a plurality of outputs of the microfluidics device measured by the first processor.

15. The computer-readable storage medium of claim 13, wherein the computer readable instructions further cause the group of processors to:
present the information output from the first processor to the second processor to a user using a display.
